(19) United States
(12) Patent Application Publication
(10) Pub. No.: US 2015/0246129 A1
(43) Pub. Date: Sep. 3, 2015

(54) PHARMACEUTICAL COMPOSITION

(71) Applicant: WOCKHARDT LIMITED, Aurangabad (IN)

(72) Inventors: Maharaj K. Sahib, Aurangabad 3 (IN); Jeetendra Kashinath Ambulge, Aurangabad 3 (IN); Gauravkumar Ramanlal Agrawal, Jalgaon 7 (IN)

(21) Appl. No.: 14/436,895

(22) PCT Filed: Apr. 19, 2013
(86) PCT No.: PCT/IB2013/053093

§ 371 Date: Apr. 19, 2015

(30) Foreign Application Priority Data
Dec. 26, 2012 (IN) 3614/MUM/2012

(51) Int. Cl. A61K 47/18; A61K 47/02; A61K 38/28

(52) U.S. Cl. CPC A61K 47/183; A61K 38/28; A61K 47/02

(57) ABSTRACT

A pharmaceutical composition comprising human insulin, analogues or derivatives thereof, at least one or more amino acids and a halogenide optionally along with one or more pharmaceutically acceptable excipient(s).

### FIELD OF THE INVENTION

The present invention provides a pharmaceutical composition comprising human insulin, analogues or derivatives thereof, at least one or more amino acids and a halogenide optionally along with one or more pharmaceutically acceptable excipient(s). The invention also provides process for preparing the pharmaceutical composition and its use in treatment of diabetes disorders.

### BACKGROUND OF THE INVENTION

Glucose is a simple sugar used by all the cells of the body to produce energy and support life. Humans need a minimum level of glucose in their blood at all times to stay alive. The primary manner in which the body produces blood glucose is through the digestion of food. When a person is not getting sufficient glucose from food digestion, glucose is produced from stores in the tissue and released by the liver. The body's glucose levels are primarily regulated by insulin. Insulin is a peptide hormone that is naturally secreted by the pancreas. Insulin helps glucose enter the body's cells to provide a vital source of energy.

Diabetes is a disease characterized by abnormally high levels of blood glucose and inadequate levels of insulin. Diabetes is a general term for disorders in man having excessive urine excretion as in diabetes mellitus and diabetes insipidus. Diabetes mellitus (DM) is a major chronic illness found in humans with many consequences. Some complications arising from long-standing diabetes are blindness, kidney failure, and limb amputations. Insulin-dependent diabetes mellitus (IDDM) accounts for 10 to 15% of all cases of diabetes mellitus.

Insulin injections are prescribed to the patients suffering from diabetes. Insulin is a natural hormone, which controls the level of the sugar glucose in the blood. In healthy people, insulin is released in blood by the pancreas as the concentration of blood glucose rises. Increased blood glucose levels, occur after meals and are rapidly compensated by a corresponding increase in insulin secretion. Insulin plays major role in converting the excess blood glucose into glycogen and storing it in liver.

Since the introduction of insulin in the 1920s, continuous strides have been made to improve the treatment of diabetes mellitus. To help avoid extreme glycaemia levels, diabetic patients often practice multiple injection therapy, whereby insulin is administered with each meal.

Insulin is a polypeptide of 51 amino acids, which are divided into 2 amino acid chains: the A chain having 21 amino acids and the B chain having 30 amino acids. The chains are connected to one another by means of two disulfide bridges. Insulin preparations have been employed for diabetes therapy for many years.

Traditionally short acting regular insulin formulations or intermediate acting insulin protamine formulations were used for treating patients with diabetes mellitus. With time, new insulin analogues and derivatives were developed. Insulin analogues and derivatives differ from human insulin at one or more than one amino acid positions and/or amino acid chain length.

A number of insulin, insulin analogs and derivatives are available in the market. The commonly used types of insulin, insulin analogs or insulin derivatives are rapid-acting Insulin analogs, short-acting insulin, Intermediate-acting insulin, Long-acting Insulin and Mixed Insulins.

Insulin Glargine (Gly(A21)-Arg(B31)-Arg(B32)-human insulin) is a long acting insulin with a prolonged duration of action. Insulin glargine is injected as an acidic, clear solution and precipitates on account of its solution properties in the physiological pH range of the subcutaneous tissue as a stable hexamer associate. Insulin glargine is injected once daily and is distinguished compared with other long-acting insulins by its flat serum profile and the reduction of the danger of nightly hypoglycemia associated therewith (Schubert-Zsilavecz et al., 2: 125-130(2001)).

Insulin analogs having a fast onset of action are described in EP0214826, EP0375437 and EP0678522. EP0124826 relates, inter alia, to substitutions of B27 and B28. EP0678522 describes insulin analogs, which have various amino acids, preferably proline, in position B29, but not glutamic acid. According to WO92/00321, insulins of this type have a prolonged action.

In addition to the duration of action, the stability of the preparation is very important for patients. Stabilized insulin formulations having increased physical long-term stability are needed in particular for preparations, which are exposed to particular mechanical stresses or relatively high temperatures.

Further, the insulin analogs, which often exist in the monomeric or dimeric form, are less stable and more prone to aggregate under thermal and mechanical stress than hexameric insulin. This makes itself noticeable in cloudiness and precipitates of insoluble aggregates (Bakaysa et al, U.S. Pat. No. 5,474,978). These higher molecular weight transformation products (dimers, trimers, polymers) and aggregates decrease not only the dose of insulin administered but can also induce irritation or immune reactions in patients. Moreover, such insoluble aggregates can affect and block the cannulas and tubing of the pumps or needles of pens.

In addition to the aggregation problem, it has been reported that insulin preparation are more prone to adsorption onto glass or plastic surfaces which then further lead to decreased stability of the final preparations. The forces involved in the adsorption reactions between proteins like insulin molecules and glass surfaces are primarily ionic amine-silanol bonding and hydrogen bonding.

Further, Chuen Shang C. Wu et. al. in ‘Adsorption of proteins onto glass surfaces and its effect on the intensity of circular dichroism spectra’ has also discussed the adsorption problem associated with apolipoproteins A-I and E, fibronectin, bovine serum albumin, insulin, and glucagon. It discloses that these molecules adsorbed most onto glass surfaces and may lead to serious errors in experimental results.

However, in order to guarantee the quality of an insulin preparation, it is necessary to avoid the formation of aggregates as well as the glass adsorption problems associated with such preparations. Numbers of attempts have been made till date to provide formulations comprising insulin that remain chemically stable for a sufficiently long period of time.

Thus, in international patent application WO98/56406, formulations stabilized by TRIS or arginine buffer have been described. U.S. Pat. No. 5,866,538 describes an insulin preparation that contains glycerol and sodium chloride in concentrations of 5-100 Mm and should have an increased stability. U.S. Pat. No. 5,948,751 describes insulin preparations having increased physical stability, which is achieved by addition of mannitol or similar sugars. The addition of excess zinc to a zinc-containing insulin solution can likewise increase the stability (J. Brange et al., Diabetic Medicine, 3: 532-536, 1986). The influence of the pH and various excipients on the stability of insulin preparations has also been described in detail (J. Brange & L. Langkjaer, Acta Pharm. Nordica 4: 149-158).

In U.S. Pat. Nos. 7,476,652 and 7,713,930 pharmaceutical formulations has been disclosed wherein that non-ionic surfactants specifically esters and ethers of polyhydric alcohols (polysorbate 20 and polysorbate 80) increase the stability of acidic insulin preparations and thus preparations can be produced which guarantee superior stability to hydrophobic aggregation nuclei for several months under temperature stress.

U.S. Pat. No. 8,263,551 discloses pharmaceutical formulations comprising insulin, insulin analogs, insulin derivatives or mixtures of the foregoing, and a salt of protamine where the protamine salt wherein protamine salt was used for increasing the physical and chemical stability of an insulin-containing formulation

U.S. Pat. No. 8,097,584 discloses that pharmaceutical polypeptide formulations having increased chemical stability can be obtained by adding ethylenediamine or salts thereof as a buffer to said formulation.

U.S. Pat. No. 4,476,118 discloses stable insulin solution comprising a preserving agent, an isotonicity agent, and a pH-buffering agent, which solution contains essentially ionized zinc.

U.S. Pat. No. 6,174,856 discloses that the stability of insulin compositions can be significantly improved by formulating the compositions using a combination of a buffer such as glycylglycine (Gly-Gly) and metal ions such as Ca2+.

U.S. Pat. No. 6,734,162 discloses a method of inhibiting aggregation of a polypeptide comprising combining the polypeptide with a buffer comprising tris(hydroxymethyl) aminomethane (TRIS) mixed with a buffering molecule that does not contain a free amine group and which mitigates the change in pH that results from the formation of carbonic acid, zinc, and a phenolic preservative for a time and under conditions effective to inhibit aggregation.

U.S. Pat. No. 6,737,401 discloses an unexpected property of the novel surfactant stabilized insulin formulations.

U.S. Pat. No. 5,866,538 discloses a pharmaceutical formulation comprising a polypeptide selected from the group consisting of human insulin, an analogue thereof, a derivative thereof, glycerol, mannitol, or glycerol & mannitol and 5 to 100 mM of a halogenide. It has been shown in the said patent that insulin preparations of superior chemical stability can be obtained in the presence of low halogenide concentrations.

US Patent Application No. 20090175840 discloses an injectable formulation comprising insulin, a diluent suitable for injection, an oxidizing agent or enzyme and a reducing agent or enzyme, with the proviso that the formulation does not contain a chitosan-glycerol phosphate hydrogel.

US Patent Application No. 20090325860 discloses an aqueous pharmaceutical formulation comprising an aqueous mixture of an insulin molecule, a solubilizing agent, a surface active agent, and a thickening agent, wherein the pharmaceutical formulation confers an ultra-rapid acting insulin profile to non-ultra-rapid acting insulin.

US Patent Application No. 20100069292 discloses a basal insulin formulation comprising a solution of recombinant human insulin at a pH between 3.5 and 4.5, preferably 3.8 to 4.2, or 7.5 to 8.5, optionally in combination with a stabilizing agent, buffering agent and precipitating agent, but not including protamine.

US Patent Application No. 20100260766 discloses methods and formulations for the stabilization of antibodies that bind insulin-like growth factor-I receptor using glycine as a stabilizers.

US Patent Application No. 20100203014 discloses glycine buffered pharmaceutical compositions of proteins suitable for nasal administration.

US Patent Application No. 20100028372 increased protein stability is obtained by: a. determining a pH at which the protein has stability at the desired temperature, b. adding to the composition at least one displacement buffer wherein the displacement buffer is glycine.

PCT Publication No. WO97/48414 discloses that insulin preparations of superior chemical stability can be obtained in the presence of glycerol and/or mannitol and rather low halogenide concentrations.

Further, Acta Pharmaceutica Nordica 4(4), 1992, pp. 149-158 discloses insulin preparations in which the sodium chloride (Halogenide) concentration has been varied in the range of 0 to 250 mM. However, the major part of the preparations, including all preparations contains a rather high amount of sodium chloride, i.e. 0.7% w/v corresponding approximately to a concentration of 120 mM.

Furthermore, several attempts have been made to provide stable insulin formulations with reduced adsorption of molecule onto the glass surfaces.

Suelter Ch et al. in ‘How to prevent losses of protein by adsorption to glass and plastic’ reports an improved procedure for reducing the loss of protein by adsorption to glass or plastic surfaces by adding glycerol in 50% concentration.

P. H. Sönksen et al. in ‘A quantitative evaluation of the relative efficiency of gelatine and albumin in preventing insulin adsorption to glass’ discloses the albumin has been found to be more effective than gelatine in preventing insulin loss from insulin containing solutions.

Several attempts to provide stable insulin formulations have been described previously. However, there still exists a need to develop formulations wherein the insulin does not undergo chemical transformation and remains stable for a sufficiently long period of time.

None of the prior arts discloses the use of amino acids in combination with a halogenide to increase the stability of the insulin preparations by reducing aggregation as well as decreasing glass adsorption.

However, the inventors of present invention have surprisingly found that the insulin preparations having better solubility and chemical stability can be obtained by using one or more amino acids in combination with a halogenide.

Further, the present inventors have found that amino acid in combination with halogenide not only prevent the aggregation of insulin molecules by reducing the surface tension but also helps in maintaining a thermodynamic and hydrodynamic balance for maintaining the stability of insulins by keeping them in their native state.

### SUMMARY OF THE INVENTION

The term ‘Insulin’ used herein includes mammalian insulin, insulin analogues or derivatives.

The term ‘Insulin analogs’ used in the present invention includes analogs of naturally occurring insulins, namely human insulin or animal insulins, which differ by substitution of at least one naturally occurring amino acid residue with other amino acid residues and/or addition/removal of at least one amino acid residue from the corresponding, otherwise identical, naturally occurring insulin. The added and/or replaced amino acid residues can also be those, which do not occur naturally.

The term ‘Insulin derivatives’ used in the present invention includes derivatives of naturally occurring insulin or of an insulin analog, which are obtained by chemical modification. The chemical modification can consist, for example, in the addition, substitution or deletion of one or more specific chemical groups to one or more amino acids. It can also involve the addition, substitution or deletion of one or more chemical groups of the peptide backbone, such as, at the amino and/or carboxyl terminus.

By ‘analogue of human insulin’ as used herein is meant human insulin in which one or more amino acids have been deleted and/or replaced by other amino acids, including non-codeable amino acids, or human insulin comprising additional amino acids, i.e. more than 51 amino acids.

By ‘derivative of human insulin’ as used herein is meant human insulin or an analogue thereof in which at least one organic substituent is bound to one or more of the amino acids.

By ‘complexing agents’ as used herein is meant a molecule that has a multiplicity of charges and that binds to or complexes with insulin compound conjugates. Examples of complexing agents suitable for use in the present invention include protamines, surfen, globin proteins, spermine, spermidine albumin, carboxylic acids, polycationic polymer compounds, cationic polypeptides, anionic polypeptides, nucleotides, and antisense. See Brange, J. Galenics of Insulin compound, Springer-Verlag, Berlin Heidelberg (1987), the entire disclosure of which is incorporated herein by reference.

By ‘polyatomic alcohol’ as used herein is meant a alcohol chosen from the group consisting of glycerol, mannitol, sorbitol, and polyols, the partial and fatty acid esters and ethers of glycerol and sorbitol being selected from the group consisting of SPAN®, TWEEN®, MYRJ®, BRIJ®, CREMOPHOR®, the polyols being selected from the group consisting of polypropylene glycols, polyethylene glycols, poloxamers, PLURONICS®, and TETRONICS®.

By ‘devoid of complexing agent’ as used herein is meant said complexing agent is present in a concentration less than 0.01% w/v of final composition.

By ‘devoid of polyatomic alcohols’ as used herein is meant said polyatomic alcohol is present in a concentration less than 0.1 mM.

By ‘preservative’ as used herein refers to the compound that can be used to prevent the growth of fungi and other microorganisms.

By ‘isotonicity agent’ as used herein is a compound that is physiologically tolerated and imparts a suitable tonicity to a formulation to prevent the net flow of water across cell membranes that are in contact with the formulation.

By ‘pH modifying agent’ as used herein refers to a combination of acid and alkali, wherein pH modifying agent is other than halogenide.

One of the aspects of the present invention provides a pharmaceutical composition comprising human insulin, analogues or derivatives thereof, at least one or more amino acids and a halogenide optionally along with one or more pharmaceutically acceptable excipient(s).

Another aspect of the present invention provides a pharmaceutical composition comprising human insulin, analogues or derivatives thereof, glycine and sodium chloride optionally along with one or more pharmaceutically acceptable excipient(s).

Another aspect of the present invention provides a pharmaceutical composition comprising insulin glargine, glycine and sodium chloride optionally along with one or more pharmaceutically acceptable excipient(s).

Another aspect of the invention provides a pharmaceutical composition comprising human insulin, analogues or derivatives thereof, at least one or more amino acids and a halogenide optionally along with one or more pharmaceutically acceptable excipient(s), wherein said composition is devoid of any complexing agent.

Another aspect of the invention provides a pharmaceutical composition comprising human insulin, analogues or derivatives thereof, at least one or more amino acids and a halogenide optionally along with one or more pharmaceutically acceptable excipient(s), wherein said composition is devoid of any polyatomic alcohol.

Yet another aspect of the present invention provides a method of using such composition to achieve a therapeutic effect, which comprises administering to a subject in need thereof an effective amount of the composition.

It is another aspect of the invention to provide a pharmaceutical composition comprising Gly(A21), Arg(B31), Arg(B32)-human insulin, glycine and a halogenide and optionally one or more other pharmaceutically acceptable excipient(s). The Gly (A21), Arg (B31), Arg(B32)-human insulin generically known as Insulin glargine.

It is the other aspect of the invention to provide a pharmaceutical composition comprising human insulin, analogues or derivatives thereof, at least one or more amino acids and a halogenide optionally along with one or more pharmaceutically acceptable excipient(s), wherein said pharmaceutically acceptable excipient(s) comprises preservative, isotonicity agent, pH modifying agent or diluent.

It is another aspect of the present invention to provide process for preparation of such composition, which comprises of:
a. preparing a solution of Insulin by dissolving insulin in acidic water for injection,
b. preparing a solution of amino acids and a halogenide along with other pharmaceutically acceptable excipients,
c. mixing the solution of step (a) and (b).

The final volume is made up with the help of water for injection (WFI). The pH is adjusted with pH modifying agents.

It is another aspect of the present invention to provide process for preparation of such composition, which comprises of:
a. preparing a solution of Insulin by dissolving insulin in acidic water for injection,
b. preparing a solution of amino acids and a halogenide along with other pharmaceutically acceptable excipients,
c. mixing the solution of step (b) with the solution of step (c),
d. preparing a solution comprising pH modifying agent,
e. adding solution of step (c) to the solution of step (d).

The final volume is made up with the help of water for injection (WFI). The pH is adjusted with pH modifying agents.

It is another aspect of the present invention to provide a method for controlling the level of glucose in a patient suffering from diabetes by administering to the subject a pharmaceutical composition of the present invention.

It is another aspect of the present invention to provide a pharmaceutical composition comprising human insulin, analogues or derivatives thereof, at least one or more amino acids and a halogenide, wherein said formulation control the level of glucose in a patient suffering from diabetes.

It is yet another aspect of the present invention to provide a method of reducing protein aggregation by formulating a pharmaceutical composition comprising human insulin, analogues or derivatives thereof, at least one or more amino acids and a halogenide optionally along with one or more pharmaceutically acceptable excipient(s).

It is yet another aspect of the present invention to provide a method of reducing protein aggregation by formulating a pharmaceutical composition comprising human insulin, analogues or derivatives thereof, at least one or more amino acids and a halogenide optionally along with one or more pharmaceutically acceptable excipient(s).

### DETAILED DESCRIPTION OF THE INVENTION

The inventors of present invention have surprisingly found that the insulin preparations having better solubility and chemical stability can be obtained by using one or more amino acids in combination with a halogenide.

Further, the present inventors have found that amino acid in combination with halogenide not only prevent the aggregation of insulin molecules by reducing the surface tension but also helps in maintaining a thermodynamic and hydrodynamic balance for maintaining the stability of insulins by keeping them in their native state.

The present invention provides a pharmaceutical composition comprising human insulin, analogues or derivatives thereof, at least one or more amino acids and a halogenide optionally along with one or more pharmaceutically acceptable excipients.

The insulin analogues used in the present invention include, but not limited to, an analogues wherein position B28 is Asp, Lys, Leu, Val or Ala; position B29 is Lys or Pro; or des(B28-B30), des(B27) or des(B30) human insulin or A21 is Gly and Arg has been added to B31 and B32; or where the amino acid residues in B28-B30 have been deleted; or where the amino acid residue at B27 has been deleted; or where the amino acid residue at B30 has been deleted. Marketed Insulin analogues include but are not limited to Insulin aspart (AspB28 human insulin), Insulin Lispro (LysB28 ProB29 human insulin), Insulin glulisine, Insulin glargine (GlyA21ArgB31ArgB32-human insulin or Gly(A21), Arg(B31), Arg(B32)-human insulin), etc.

The insulin derivatives used in the present invention include, but not limited to, B29-Nε-myristoyl-des(B30) human insulin, B29-Nε-palmitoyl-des(B30) human insulin, B29-Nε-myristoyl human insulin, B29-Nε-palmitoyl human insulin, B28-Nε-myristoyl LysB28 ProB29 human insulin, B28-Nε-palmitoyl LysB28 ProB29 human insulin, B30-Nε-myristoyl-ThrB29 LysB30 human insulin, B30-Nε-palmitoyl-ThrB29 LysB30 human insulin, B29-Nε-(N-palmitoyl-γ-glutamyl)-des(B30) human insulin, B29-Nε-(N-lithocholyl-γ-glutamyl)-des(B30) human insulin, B29-Nε-carboxyheptadecanoyl)-des(B30) human insulin and -B29-Nε-(ω-carboxyhept adecanoyl) human insulin, 29B-[N6-(Oxo-tetradecy)-1-lysine]-(1A-21A),(1B-29B)-insulin (human) (insulin detemir).

In one aspect of the present invention, there is provided pharmaceutical composition comprising human insulin, analogues or derivatives thereof, at least one or more amino acids and a halogenide optionally along with one or more pharmaceutically acceptable excipients, wherein the human insulin, analogues or derivatives thereof is selected from one or more of recombinant human insulin, Insulin NPH, Insulin Lispro, Insulin Lispro Protamine, Insulin Glulisine and Insulin Aspart, Insulin Aspart Protamine, Insulin glargine, and insulin detemir.

In one aspect of the present invention, there is provided a pharmaceutical composition comprising human insulin, analogues or derivatives thereof, glycine and sodium chloride optionally along with one or more pharmaceutically acceptable excipient(s).

In one aspect of the present invention, there is provided a pharmaceutical composition comprising insulin glargine, glycine and sodium chloride optionally along with one or more pharmaceutically acceptable excipient(s).

The amino acid in pharmaceutical composition of the invention is present in a concentration from about 12 mM to about 60 mM.

The pH of the pharmaceutical composition of the invention is between about 3.5 to about 7.0.

The pH of the pharmaceutical composition of the invention is between about 3.5 to about 4.5.

In one aspect of the present invention, there is provided a pharmaceutical composition comprising human insulin, analogues or derivatives thereof, glycine and sodium chloride optionally along with one or more pharmaceutically acceptable excipient(s), wherein the human insulin, analogues or derivatives thereof is selected from one or more of recombinant human insulin, Insulin NPH, Insulin Lispro, Insulin Lispro Protamine, Insulin Glulisine and Insulin Aspart, Insulin Aspart Protamine, Insulin glargine, and insulin detemir.

In one aspect of the present invention, there is provided a pharmaceutical composition comprising insulin glargine, glycine and sodium chloride optionally along with one or more pharmaceutically acceptable excipient(s), wherein the human insulin, analogues or derivatives thereof is selected from one or more of recombinant human insulin, Insulin NPH, Insulin Lispro, Insulin Lispro Protamine, Insulin Glulisine and Insulin Aspart, Insulin Aspart Protamine, Insulin glargine, and insulin detemir.

In one aspect of the present invention, there is provided a pharmaceutical composition comprising human insulin, analogues or derivatives thereof, at least one or more amino acids and a halogenide optionally along with one or more pharmaceutically acceptable excipient(s), wherein said composition is devoid of any complexing agent.

In one aspect of the present invention, there is provided a pharmaceutical composition comprising human insulin, analogues or derivatives thereof, at least one or more amino acids and a halogenide optionally along with one or more pharmaceutically acceptable excipient(s), wherein said composition is devoid of any polyatomic alcohol.

In one aspect of the present invention, there is provided pharmaceutical composition comprising human insulin, analogues or derivatives thereof, at least one or more amino acids and a halogenide, wherein the pH of the composition is from about 3.5 to about 4.5 optionally along with one or more pharmaceutically acceptable excipients, wherein the human insulin, analogues or derivatives thereof is selected from one or more of recombinant human insulin, Insulin NPH, Insulin Lispro, Insulin Lispro Protamine, Insulin Glulisine and Insulin Aspart, Insulin Aspart Protamine, Insulin glargine, and insulin detemir.

In one aspect of the present invention, there is provided a pharmaceutical composition comprising human insulin, analogues or derivatives thereof, glycine and sodium chloride, wherein the pH of the composition is from about 3.5 to about 4.5 optionally along with one or more pharmaceutically acceptable excipient(s), wherein the human insulin, analogues or derivatives thereof is selected from one or more of recombinant human insulin, Insulin NPH, Insulin Lispro, Insulin Lispro Protamine, Insulin Glulisine and Insulin Aspart, Insulin Aspart Protamine, Insulin glargine, and insulin detemir.

In one aspect of the present invention, there is provided pharmaceutical composition comprising human insulin, analogues or derivatives thereof, at least one or more amino acids and a halogenide, wherein the pH of the composition is from about 3.5 to about 4.5 optionally along with one or more pharmaceutically acceptable excipients.

In one aspect of the present invention, there is provided pharmaceutical composition comprising insulin glargine, at least one or more amino acids and a halogenide, wherein the pH of the composition is from about 3.5 to about 4.5, optionally along with one or more pharmaceutically acceptable excipients.

The amino acids used for the present invention includes but are not limited to glycine, arginine, histidine, lysine, serine, threonine, aspartic acid, glutamic acid, serine, threonine, glutamine, asparagine, alanine, isoleucine, leucine or salts thereof.

The amino acids used for the present invention include glycine, arginine, histidine or salts thereof. More specifically, the amino acid used is glycine.

The halogenide used for the present invention include an alkali or alkaline earth metal halogenide, more preferably a chloride such as sodium chloride.

The amino acids in combination with halogenide used in the present invention not only prevent the aggregation of insulin molecules by reducing the surface tension but also helps in maintaining a thermodynamic and hydrodynamic balance for maintaining the stability of insulins by keeping them in their native state.

The pharmaceutically acceptable excipients include, but are not limited to, preservatives, isotonicity agents, pH modifying agents or diluent.

The ‘preservative’ as used herein include, but are not limited to, benzoic acid, butylparaben, ethyl paraben, methyl paraben, propylparaben, sodium benzoate, sodium propionate, benzalkonium chloride, benzethonium chloride, benzyl alcohol, cetypyridinium chloride, chlorobutanol, phenol, phenylethyl alcohol, metacresol or combinations thereof.

An ‘isotonicity agent’ is a compound, such as glycerin, are commonly used for such purposes at known concentrations. Other possible isotonicity agents include salts, e.g., sodium chloride, dextrose, or lactose.

An ‘pH modifying agent’ as used herein can be selected form the group comprising of o-phosphoric acid, citric acid, acetic acid, succinic acid, lactic acid, gluconic acid, tartaric acid, 1,2,3,4-butane tetracarboxylic acid, fumaric acid or malic acid. Alkali is selected form the group comprising of sodium hydroxide, potassium hydroxide, sodium hydroxide, ammonium hydroxide, magnesium oxide, calcium hydroxide, calcium carbonate, magnesium carbonate, magnesium aluminum silicates, diethanolamine, monoethanolamine, sodium carbonate, sodium bicarbonate or triethanolamine.

Typically the insulin is dissolved or dispersed in a diluent to provide the insulin in a liquid form. Suitable diluents include, but are not limited to, water, buffered aqueous solutions, dilute acids, vegetable or inert oils for injection organic hydrophilic diluents, such as monovalent alcohols, and low molecular weight glycols and polyols (e.g. propylene glycol, polypropylene glycol, glycerol, and butylene glycol).

In the present invention, there is provided a process for preparation of an insulin composition, wherein said process comprises of:
a. preparing a solution of insulin by dissolving insulin in acidic water for injection,
b. preparing a solution of amino acids and a halogenide along with other pharmaceutically acceptable excipients,
c. mixing the solution of step (a) and (b).

The final volume is made up with the help of water for injection (WFI). The pH is adjusted with pH modifying agents.

In another aspect of the present invention, there is provided a process for preparation of an insulin composition involving the following steps:
a. preparing a solution of Insulin by dissolving insulin in acidic water for injection,
b. preparing a solution of amino acids and a halogenide along with other pharmaceutically acceptable excipients,
c. mixing the solution of step (b) with the solution of step (c),
d. preparing a solution comprising pH modifying agent,
e. adding solution of step (c) to the solution of step (d).

The final volume is made up with the help of water for injection (WFI). The pH is adjusted with pH modifying agents.

In another aspect of the present invention, there is provided a method for treating diabetes comprising administering a pharmaceutical composition of the present invention to the patient in need thereof.

In another aspect of the present invention, there is provided a method for controlling the level of glucose in a patient suffering from diabetes comprising administering to the patient a pharmaceutical composition of the present invention.

In another aspect of the present invention, there is provided a pharmaceutical composition comprising human insulin, analogues or derivatives thereof, at least one or more amino acids and a halogenide, wherein said composition control the level of glucose in a patient suffering from diabetes.

In another aspect of the present invention, there is provided a method of reducing protein aggregation by formulating a pharmaceutical composition comprising human insulin, analogues or derivatives thereof, at least one or more amino acids and a halogenide optionally along with one or more pharmaceutically acceptable excipient(s).

The example given below serves to illustrate embodiments of the present invention. However it does not intend to limit the scope of present invention.

### Example-1

TABLE 1
Pharmaceutical Composition

| S. No. | Ingredients | Quantity (mg/ml) |
|---|---|---|
| 1 | Insulin Glargine | 100 IU |
| 2 | Metacresol | 1.5-4.5 |
| 3 | Sodium Chloride | 3-7 |
| 4 | Zinc as Zinc chloride | 0.01-0.06 |
| 5 | Glycine | 1.5-7 |
| 6 | Sodium Hydroxide | Q.S. |
| 7 | Hydrochloric Acid | Q.S. |
| 8 | Water for injection | Q.S. |

### Procedure:

The pharmaceutical composition described in Example 1 was prepared according to the procedure detailed below:

### Step 1: Preparation of API Solution

Accurately weighed insulin glargine was prepared by dissolving zinc-containing crystals of Insulin Glargine in water for injection with the help of few μL of 1M HCl. The endogenous zinc level was supplemented by adding appropriate volume of zinc chloride solution (1% w/v) to achieve final concentration of 30 μg/100 IU.

### Step 2: Preparation of Buffer Solution

Buffer solution was prepared by dissolving metacresol, sodium chloride and glycine to get final concentration as 25 mM Metacresol, 75 mM Sodium Chloride and 50 mM Glycine.

### Step 3: Preparation of Final Solution

Both API and Buffer solution were diluted to final concentrations after mixing and pH adjustment to 4.0±0.2 with 1M HCl or 1M NaOH.

### Example-2

TABLE 2
Pharmaceutical Composition

| S. No. | Ingredients | Quantity per mL |
|---|---|---|
| 1. | Insulin Glargine | 100 IU |
| 2. | Metacresol | 1.5-4.5 mg/mL |
| 3. | Sodium Chloride | 1.0-7.0 mg/mL |
| 4. | Zinc as Zinc chloride | 0.01-0.06 mg/mL |
| 5. | Glycine | 1.5-9.0 mg/mL |
| 6. | Sodium Hydroxide | q. s. to adjust pH 4.0 |
| 7. | Hydrochloric Acid | q. s. to adjust pH 4.0 |
| 8. | Water for injection | q. s. to 1.0 mL |

### Procedure:

The pharmaceutical composition described in Example 2 was prepared according to the procedure detailed below:

### Step 1: Preparation of API Solution

Accurately weighed insulin glargine solution was prepared by dissolving zinc-containing crystals of insulin glargine in water for injection with the help of few μL of 1M HCl. The endogenous zinc level was supplemented by adding appropriate volume of zinc chloride solution (1% w/v) to achieve final concentration of 30 μg/100 IU.

### Step 2: Preparation of Buffer Solution

Buffer solution was prepared by dissolving metacresol, sodium chloride and glycine to get final concentration as 25 mM metacresol, 50 mM sodium chloride and 100 mM glycine.

### Step 3: Preparation of Final Solution

Both API and buffer solutions were diluted to final concentrations after mixing and pH adjustment to 4.0±0.2 with 1M HCl or 1M NaOH.

After sterile filtration, these preparations were introduced into vials and subjected to stability testing.

### Example-3

### Stability Testing

The pharmaceutical composition according to Example 2 of the present inventions were subjected to Real Time stability conditions at 5° C.±3° C. and Forced Degradation study at 55° C.±2° C.

### A. Real Time Stability Study:

Samples were withdrawn initially and after 3 months stored at 5° C.±3° C. and subjected to HPLC analysis. Results are reproduced in Table 3.

TABLE 3
Real Time Stability Studies

| | | Results | |
|---|---|---|---|
| | | | 3 Months |
| Tests | Acceptance Criteria | Initial | (5° C. ± 3° C.) |
| Description | A clear, colourless or | A clear and | A clear and |
| | almost colourless | colourless | colourless |
| | liquid. | liquid | liquid |
| pH | Between 3.7 and 4.3 | 4.08 | 4.07 |
| Assay of | Not less than 95.0 IU | 103.59 IU/mL | 104.45 IU/mL |
| Insulin | and not more than | | |
| Glargine | 105.0 IU per mL | | |
| Related | Not more than 6% | 1.58% | 1.47% |
| Impurities | | | |
| Limit of High | Not more than 2.0% | 0.033% | 0.05% |
| Molecular | | | |
| Weight | | | |
| Proteins | | | |
| Preservative | Between 2.43 to 2.97 | 2.60 mg/mL | 2.68 mg/mL |
| (m-Cresol) | mg per mL | | |
| Content | | | |

### B. Forced Degradation Study at 55° C.±2° C.

Samples were withdrawn initially and after 2 days incubated at 55° C.±2° C. and subjected to HPLC analysis.

TABLE 3
Forced Degradation Stability Studies

| | | Results | |
|---|---|---|---|
| Tests | Acceptance Criteria | Initial | 2 Days (55° C. ± 2° C.) |
| Description | A clear, colourless or | A clear and | A clear and |
| | almost colourless | colourless | colourless |
| | liquid. | liquid | liquid |
| pH | Between 3.7 and 4.3 | 4.08 | 4.07 |
| Assay of | Not less than 95.0 IU | 103.59 IU/mL | 101.08 IU/mL |
| Insulin | and not more than | | |
| Glargine | 105.0 IU per mL | | |
| Related | Not more than 6% | 1.58% | 2.42% |
| Impurities | | | |
| Limit of High | Not more than 2.0% | 0.033% | 0.476% |
| Molecular | | | |
| Weight | | | |
| Proteins | | | |
| Preservative | Between 2.43 to 2.97 | 2.60 mg/mL | 2.50 mg/mL |
| (m-Cresol) | mg per mL | | |
| Content | | | |

As evident from the results of stability studies, the composition of Example 2 comprising Insulin glargine, glycine and a halogenide along with other excipients was found to be stable.

1. A pharmaceutical composition comprising human insulin, analogues or derivatives thereof, at least one or more amino acids and a halogenide optionally along with one or more pharmaceutically acceptable excipient(s).

2. The pharmaceutical composition according to claim 1, wherein the human insulin, analogues or derivatives thereof is selected from one or more of recombinant human insulin, Insulin NPH, Insulin Lispro, Insulin Lispro Protamine, Insulin Glulisine and Insulin Aspart, Insulin Aspart Protamine, Insulin glargine, and insulin detemir.

3. The pharmaceutical composition according to claim 2, wherein the human insulin, analogues or derivatives thereof is Insulin glargine.

4. The pharmaceutical composition according to claim 1, wherein the amino acid is selected form the group comprising of glycine, arginine, histidine, lysine, serine, threonine, aspartic acid, glutamic acid, serine, threonine, glutamine, asparagine, alanine, isoleucine, leucine or salts thereof.

5. The pharmaceutical composition according to claim 4, wherein the amino acid is glycine.

6. The pharmaceutical composition according to claim 5, wherein the amount of glycine is from about 12 mM to about 60 mM.

7. The pharmaceutical composition according to claim 1, wherein halogenide is an alkali or alkaline earth metal halogenide.

8. The pharmaceutical composition according to claim 1, wherein halogenide is sodium chloride.

9. A pharmaceutical composition comprising insulin glargine, glycine and sodium chloride optionally along with one or more pharmaceutically acceptable excipient(s).

10. A pharmaceutical composition comprising human insulin, analogues or derivatives thereof, at least one or more amino acids and a halogenide optionally along with one or more pharmaceutically acceptable excipient(s), wherein said composition is devoid of any complexing agent.

11. A pharmaceutical composition comprising human insulin, analogues or derivatives thereof, at least one or more amino acids and a halogenide optionally along with one or more pharmaceutically acceptable excipient(s), wherein said composition is devoid of any polyatomic alcohol.
